(19) United States
(12) Patent Application Publication
(10) Pub. No.: US 2020/0146883 A1
(43) Pub. Date: May 14, 2020

(54) Wearable Thermal Device with Port

(71) Applicant: Stanley A. Sansone, Houston, TX (US)

(72) Inventor: Stanley A. Sansone, Houston, TX (US)

(21) Appl. No.: 16/255,511

(22) Filed: Jan. 23, 2019

Related U.S. Application Data
(60) Provisional application No. 62/757,251, filed on Nov. 8, 2018.

(51) Int. Cl. A61F 7/08; A61F 7/10; A61F 7/00; A61B 5/107

(52) U.S. Cl. CPC A61F 7/086; A61F 7/103; A61F 7/0085; A61F 2007/0045; A61F 2007/0096; A61F 2007/0002; A61F 2007/0036; A61B 5/1077

(57) ABSTRACT

Wearable device is disclosed to quickly deliver thermal treatment. port. A separate material or component is later added through a port at point of injury or a medical event. Wearable thermal device comprises: at least one containment pack of selected shape; containment pack(s) contains a selected amount of first material (preloaded component); at least one port is attached to containment pack(s) configured to accept a second material (second component); and said second material is added through said at least one port to create a chemical reaction when combined with said first material. In one embodiment, first material needed to create an endothermic reaction is a selected amount of ammonium nitrate contained within wearable hypothermic treating device. A port or valve is configured to accept water. When a selected amount ammonium nitrate is combined with water a selected amount of thermal mass is created.

### FIELD OF THE INVENTION

This invention relates generally to wearable thermal devices fillable through at least one port. Wearable thermal devices are used in emergency, through post emergency treatments, and/or may contain sensors used during the medical event.

### BACKGROUND OF THE INVENTION

Most thermal or therapeutic treatments applied to the human body tend to be thermal packs, heating pads, ice packs, instant cold packs, cooling blankets or electric blankets. These other treatments are intended to lay on a body part though straps or other fastening systems or mechanisms have been devised to hold these thermal treating devices to the body, but lack novel elements of this invention.

Adam U.S. Pat. No. 5,864,880 forms the basis of an insulated liquid delivery system. Adam's removable bladder is inserted into the back of a jersey. The bladder is intended to be carried as a canteen or flask when biking or hiking and to provide accessible drinking fluids through a straw that is within and through to the bottom of bladder. This wearable device is not intended to cover a part of the body to deliver thermal treatments to said body part. In fact Adam teaches the use of insulation between removable bladder and person wearing jersey with removable bladder. Adam's jersey cannot deliver thermal therapeutic treatment.

U.S. Pat. No. 9,132,030 by Koudelka shows the state of the therapeutic wrap art. All materials inside said thermal therapeutic wrap are called thermoconductive materials. Thermoconductive materials are disposed into pack(s) and sealed into chambers which include; basmati rice, buckwheat, water, or chemically engineered material such as commercially available gel packs for heating and cooling. No openings and/or ports are used to have easy access to contents of chambers. Some of the issues or problems with this state of the art of Koudelka include but not limited to: apparatus and material create heavy and bulky shipping; because of more weight and bulk, shipping costs are increased; packaging costs increase; there are direct and indirect associated environmental costs with transportation and waste, at times prepackaged thermalconductive materials need to be replaced leading to addition to landfills and increasing costs for the customer; because contents cannot be simply refreshed, and/or old thermalconductive materials cannot be replenished.

Elkins U.S. Pat. No. 8,900,170 provides a therapeutic cap with fluid space and air bladder. Fluid space and an air bladder with multiple access openings. These openings are separate openings and function as: inlet to bring cool fluids into cap which is located on one side of the cheek; outlet for removing fluids from cap is located on other cheek; and air bladder coupled to external machine to fill air bladder with elevated pressure gas. Elkins shows separate inlets and outlets into a volume. All these cap bladder openings require special pressured valves or connections that only function in conjunction with a large, bulky pump, and heat exchanger. The Elkins system is expensive and cap is a highly irregular shape that is stitched together using cords making it difficult to handle. A disadvantage is that material cannot be poured into volume or bladder but rather needs to be pumped into cap, which requires hoses and machinery. Another disadvantage is its complexity where pressure is maintained with a separate bladder filled with pressurized air.

Koudelka, Allen et al, and Elkins are concerned about temperature ranges and desired temperatures but neglect to place simple temperature sensors to ensure safety and effectiveness. Their control of temperature uses no sensors; no biosensors; nor any feedback sensor or sensors. These devices lack any ability to sense patient's biological characteristics that may be crucial to treatment.

Gilstad et al, U.S. Pat. No. 7,716,761 describes the multiuse port, which comprises a simple window allowing access to a person being supported by a positioning device.

Hu et al U.S. Pat. No. 9,492,301 a circumferential walker is typical of aircasts or air casts, which contain bladders or inflation tubes that have an inflation port to inject air into said bladder or inflation tubes. Hu cannot deliver effective thermal treatments commonly needed in injuries or surgeries. Hu's pressured inflation requires hoses, coupling and external air pressure machine which results in; extra weight, larger size, additional equipment, and increased manufacturer and consumer costs.

Lennox et al U.S. Pat. No. 8,454,671 a Rapid Cool System directs agitated cold water under pressure to the scalp or pads through a 2 port system. Like Hu Rapid Cool uses a series of tubes, fluid channels and fluid jets hole size is 0.1-to-0.76 millimeters built within a complex manifold structure. Objects like sand, organic matter or materials from chemical reaction will plug certain areas. Lennox uses up to 3 separate fittings for pressurized fluid exchange. A fluid inlet fitting is connected to an inlet tube and infusion manifold; and a separate and up to 2 fluid outlet fittings is or are connected to an outlet fitting, fluid outlet tube, air manifold, swivel type outlet connector, and aspiration manifold. Lennox further suggests that the inlet and outlets may be “located in proximity” to one another but still separate. In addition, Lennox teaches the use of additional openings such as pressure relief valves. Like Lennox, Johnson et al U.S. Pat. No. 5,314,455 uses 2 separate ports, openings, or vents to control already thermally treated fluids to place in apparatus. Use of such a system like Lennox requires elaborate and external source of thermal energy be transported into the apparatus to initiate cooling.

Lennox uses a body temperature sensor not attached to the device but rather inserted within a bladder or ear of a patient. A disadvantage is the body temperature sensor has to be attached separately and does not provide information about device operation and, in particular, the body part of interest.

Johnson et al U.S. Pat. No. 5,314,455 uses an opening connection that is coupled to a hose that extends through thermal compress cuff for admitting cold liquid under pressure into compartments. Johnsons said opening is connected to a neck and tubes whose inside diameter is 5/16″. Tubes also act as internal syphon which function; as all of the fluid can be completely drained from the bottom much like Hu. Johnson teaches draining of thermal fluid is important for rechilling the fluid warmed during extended therapy. A major disadvantage is Johnson opening is directly connected to the compartments through a maze of necks and tubes making filling and draining difficult. Johnson structure also requires a hose to make a connection creating: extra structure; more weight and size; and increased costs. Simply pouring fluids into Johnsons maze of tukbes and compartments is not possible, thus requiring a pump.

Kaib et al U.S. Pat. No. 9,861,806 similarly show prepackaged gel deployment receptacles in a wearable defibrillator device and has no port or opening for any ability for fluid or gas transporting mechanism for thermal therapy. Kaib's never uses the terms thermal, hot, heat, cool nor cold. Kaib's connection port is “an SMS may take power from the mobile systems through a port such as a USB port”.

### SUMMARY OF INVENTION

This invention relates to an apparatus that can provide therapeutic thermal treatment to a part of the body and may be filled by end user or customer at a selected time. An example is a wearable thermal device of a selected shape and selected volume is shipped to a customer without thermally treatable material. Advantages include: less shipping weight; less shipping size and packaging; less shipping costs; less packaging costs; less environmental costs, both direct and indirect; providing the customer ability to refresh contents of wearable device thereby decreasing waste. A novel feature is: said selected volume is use to provide a selected amount of thermal energy; and/or selected volume is controlled by selectively sealed portions of device which also functions to control flow and pooling.

This invention relates to an apparatus to deliver thermal therapeutic treatment that: wraps around a part of the body for thermal treatment; completely covers an appendage; and/or, completely cover a body. Such wraps may include, but not limited to: appendage; neck; or thorax. Wearable thermal device may act like a bag with or without a long sleeve to completely cover an appendage. Wearable thermal device may be a body bag to control a desired temperature of a whole body.

This invention relates generally to an apparatus port to assist in the filling of a wearable thermal device. An advantage is the wearable thermal device may be filled and refilled by the customer or purchaser of the product and not by the manufacturer. Advantage is reduction of shipping and packaging costs, and labor.

An important characteristic of the present invention is the immediate application of thermal treatments from a wearable thermal device in which a chemical reaction is initiated when at least another and necessary material is separately added through port at a selected time. It is well known that the quick application of selected thermal treatments can be a therapeutic and life-saving during a medical event. This eliminates the extra weight and size of wearable thermal device when second component is not carried in or on wearable thermal device.

An advantage is the wearable device may be safer to carry. Since the second and needed component or material is not present in the system, accidental activation of thermal chemical reaction and the release of thermal energy are eliminated. For example, a thermal device like an instant cold pack may contain a separate package of water. Eliminating the water from the device reduces its accidental activation and subsequent undesirable timing of an endothermic reaction.

This invention relates generally to an apparatus for the immediate application of cooling treatments to different parts of an injured body. It is well known that the quick application of selected hypothermic treatments can be a therapeutic and life-saving event. Quick application can reduce the effects of an injury and in many instances: arrest the injury from worsening; decreases cellular permeability; and vasoconstriction. An important characteristic of the present invention is that a chemical reaction within wearable thermal device is the most immediate and targeted application possible. Surgical facilities already overloaded with equipment would not need equipment to cool a patients appendage or body part as this function is self-contained within wearable thermal device. Even if additional cooling is required a much smaller cooling unit within the surgical unit would be a great advantage over the prior art.

This invention relates generally to an apparatus for the quick application of warming or hot treatments to different parts of an injured body. The timely application of form fitting warming therapy compress device reduces the effects of hypothermic injury, aids in the healing process after surgery, and promotes blood flow and vasodilation. Chemical reaction within an apparatus to apply thermal treatments generally is the fastest and quickest application possible and is an important characteristic of the present invention.

Another advantage is multiuse port may be, or consist of, at least one valve. The use of at least one valve also allows for: the. At least one valve may be used to: control pressure within wearable thermal device; introduce into wearable thermal device chemical or material needed to activate chemical thermal reaction; introduce thermally treatable material; introduce thermally treated material; and/or minimize leakage.

An advantage of invention is to provide a multiuse port where 2 types of thermally treatable material introduction are provided; non-pressure and pressure. Multiuse port may be simply used to allow the pouring or introduction of thermally treatable material into wearable thermal device, described as non-pressured introduction. Same multiuse port may consist of at least one valve where introduction of thermally treatable material may be injected into wearable thermal device, described as pressured introduction. A multiuse port provides optional usability for different situations. These different situations include: tactical combat zone; emergency scenes; and/or surgical setting. Having a single wearable thermal device used throughout various medical situations offers advantages including but not limited to: saves manufacturing costs; save lives; minimizes training; and other advantages.

This invention relates generally to an apparatus comprising a port with at least 2 valves to extend thermal treatment. Valves allow fluids or gases to circulate throughout wearable thermal device. A particular embodiment of invention relates an apparatus for the extended and longevity of thermal treatment. For example, when an endothermic derived cooling treatment expires or warms the valves of the port may be connected to external cool treating machine where material is thermally conditioned and then circulated through port and into wearable thermal device. Thermally treated materials generally refers to liquids and/or gases that are treated, conditioned, heated and/or cooled externally and then introduced into wearable thermal device. This creates a novel apparatus that can be activated to deliver thermal treatment within seconds and then last indefinitely.

Another advantage is to provide a multiuse port with at least one screen or filter to control flow. Control may mean limiting contaminants from being transported into wearable device as in a filter, where contaminants may be: material that can puncture wearable device; improper material; and/or material that can cause blockage within wearable device. Control may mean to protect valves from blockage that may get blocked from contaminants and/or unspent materials.

Another novel feature is to provide screens and/or filters that may be used to provide uniform distribution and/or application of contents expelled from wearable thermal device or containment pack. This novel feature provides a second use capability for wearable thermal device. When contents such as those from a used up chemical reaction of ammonia nitrate and water can be expelled or projected from wearable thermal device said screen can aid the novel distribution of contents onto the ground for the purpose to fertilize fields and grow food.

Another advantage is to provide barriers to distribute desired thermal treatments evenly throughout the said wearable device. Barriers or volume deflectors have been used to control the flow of externally thermal treated material throughout the wearable thermal device. A novel feature is the use of a semi-permeable volume deflectors where instead of a single barrier or fence line said semi-permeable volume deflectors can be made of small sealed portions of containment pack organized into lines and provide novel flow to eliminate blockage in different portions of wearable device. Barriers or volume deflectors are also used to: control volume, a selected volume, or a selected amount of volume in a novel way. Volume can be used to determine the amount of thermal mass energy that can be transferred to reach desired target temperatures of appendage or body part; and/or create compartments for pooling or targeting of thermal energy.

Another novel feature of invention is ability to control temperature and amount of heat to be transferred. Control of temperatures may consist of selecting an amount of each component needed to create a certain amount of thermal energy created by a thermal reaction. The amount of heat to be transferred is controlled by selecting a volume of thermal energy consistent with target temperatures.

It is the intent of the current invention to provide onboard sensors. An advantage of this novel feature is the coupling application. A novel feature is the superior and consistent coupling of biosensors, and/or providing body part or body diagnostics during thermal therapeutic treatment. Onboard sensors are connected or attached to wearable thermal device and/or containment pack, and may include, but not limited to: device sensors to measure system or device, performance or diagnostics; and/or biosensors to measure physical and biological characteristics of appendage or body part covered by wearable thermal device. Onboard sensors may be placed in a separate layer, called a sensor layer. Examples include but not limited to: temperature sensors for the device connected and unconnected; sensors to measure temperature of appendage and/or body part; electrical sensors as in EEG electroencephalogram; and or optical sensors as in PPG, photoplethysmography. An example is a wearable device consisting of a single layer of multiple PPG sensors with the intent to measure in a 3 dimensional (3D) analysis: blood flow; blood pressure; cellular pressure; and/or oxygen levels of appendage or body part. Other advantages include, but not limited to: creation of a 3D volume; images; and/or video for detailed observation of appendage or body part function.

This invention relates generally to an apparatus which uses biosensors to control thermal regulation. Such control may include, but not limited to: biosensors communicate with at least one valve with aperture or valve control; biosensors may communicate through port junction to external thermal treating machine, where said machine may control temperature and/or pressure flow; and/or some combination.

Another advantage is to provide a port consisting of 2 valves of different sizes and/or flow characteristics. Different valves sizes may maintain and/or control a selected pressure within or inflatedness of wearable device. For example, the flow of fluids and/or gases: into a wearable device from an inlet valve which is larger and/or greater than; flow out of the wearable device through an outlet valve which is smaller than inlet valve. Inflatedness of wearable thermal device can be controlled by external machine or special sealing of volume deflectors around out take valves.

### BRIEF DESCRIPTION OF THE DRAWINGS

FIG. 1 shows a wearable thermal device with port and sensor.

FIG. 2 shows a foot wearable thermal device with port.

FIG. 3A shows an appendage wearable thermal device configured as a bag.

FIG. 3B shows a cross section of appendage wearable thermal device of FIG. 3A.

FIG. 4 shows multiuse port assembly.

FIG. 5 shows cross sectional view of multiuse port body.

FIG. 6 shows a top view into body of multiuse port body.

FIG. 7 shows multiuse port assembly.

FIG. 8 shows multiuse port in combination with a foot wearable thermal device.

FIG. 9 shows multiuse port in combination with a head wearable thermal device.

FIG. 10 shows wearable thermal device with biosensors.

### DETAILED DESCRIPTION OF THE PREFERRED EMBODIMENTS

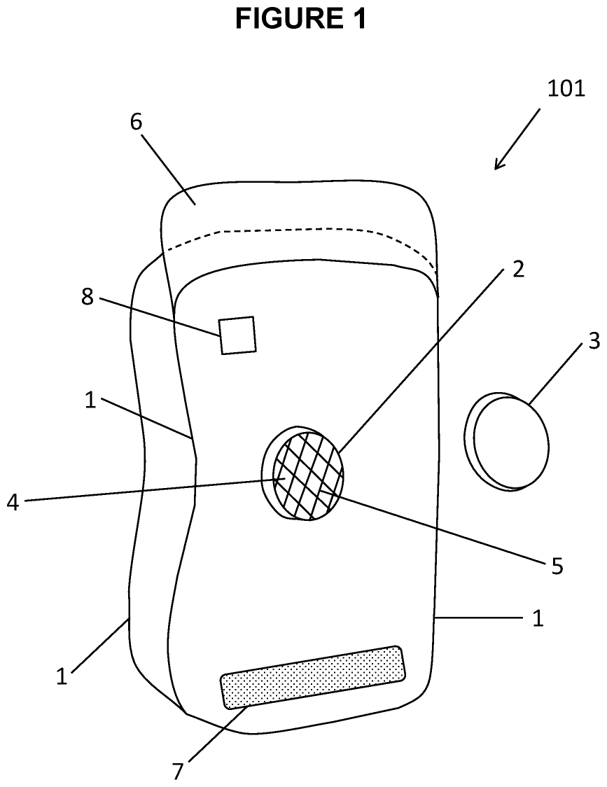

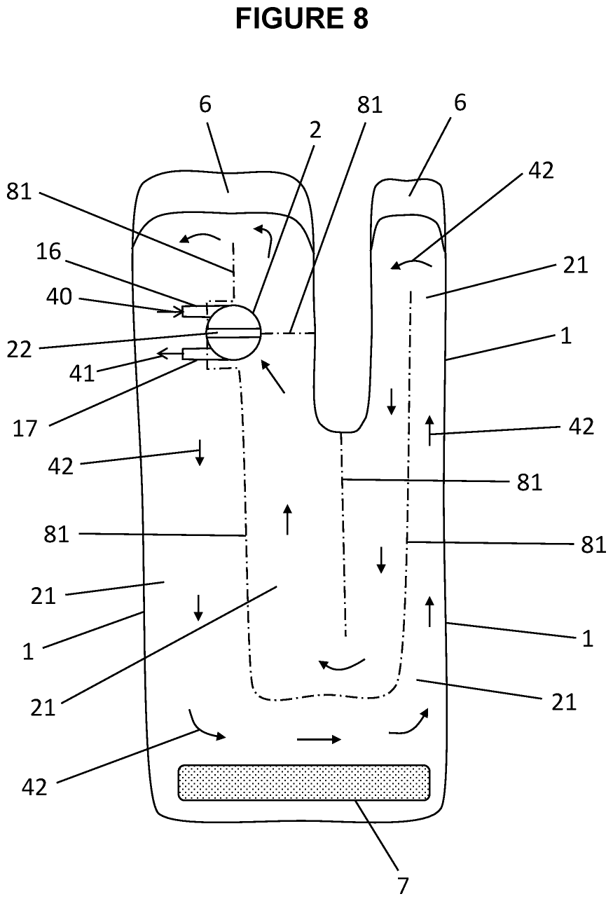

FIG. 1 shows a wearable thermal device 101 consisting of containment pack 1 and port body 2. Port body 2 is attached to containment pack 1 and provides port opening 4 into volume 21 (see FIG. 8, 9). Containment pack 1 may be, including but not limited to: the wearable thermal device; have layers attached such as insulation, skin protection, and/or barriers; and/or a bag or bladder which may be inserted into said wearable thermal device. Containment pack 1 consists of: selected shape; selected internal volume; and/or selected internal configuration. Containment pack 1 is fillable with thermally treatable material. Screen 5 may be used for functions such as, but not limited to: provides a filter to limit objects from entering and harming the containment pack 1; limits spillage of preloaded component out of containment pack 1; protect components of multiuse port; and/or provides a selected projection of contents from within containment pack 1 similar to a shower head. Screen 5 may be curved or channeled for a desired application of projection when contents are expelled from wearable thermal device and/or containment pack 1. Closing cap 3 seals contents of containment pack 1 to prevent leakage. Fastening system may consist of fastening flap 6 and fastening counterpart 7. Fastening flap 6 and fastening counterpart 7 allow wearable thermal device to: stay wrapped around a limb, spine, thorax or other body part; and/or hold appendage wearable thermal device in place. Fastening flap 6 and fastening counterpart 7 may consist of: zipper; hook and loop; or other fastening system. At least one sensor 8 is attached to containment pack 1 and may consist of at least one temperature sensor or other sensors. Temperature sensors may function to measure: temperature of materials within containment pack 1; and/or temperature of body part covered by wearable thermal device. Sensors may be used to: body part monitoring and measurement; imaging; determine treatment and/or adjust treatment; and/or provide feedback for autonomous regulation of treatment.

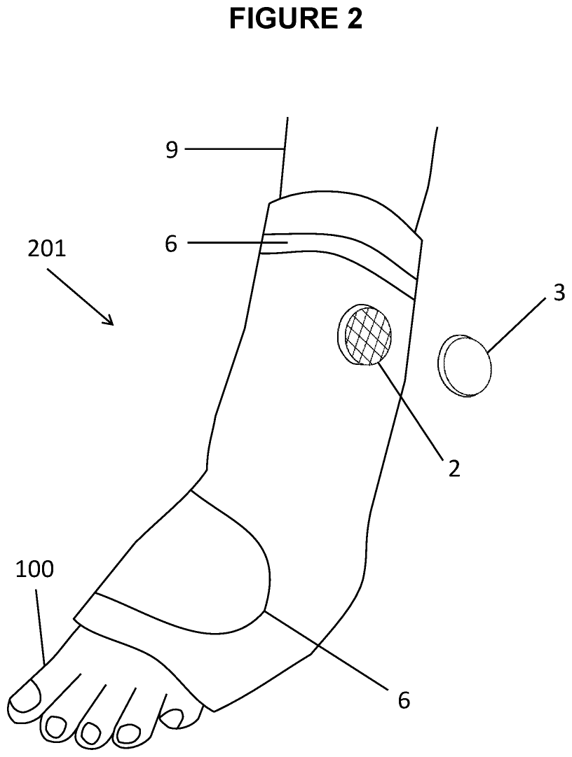

FIG. 2 shows a wrapping type foot wearable thermal device 201 with port body 2 and closing cap 3. Containment pack 1 wraps around a selected portion of a leg 9 and to the toes 10. Fastening flap 6 and fastening device 66 may have closing counterparts attached to containment pack 1, and/or a cord and lock mechanism to secure wearable thermal device around a foot and selected length of leg 9.

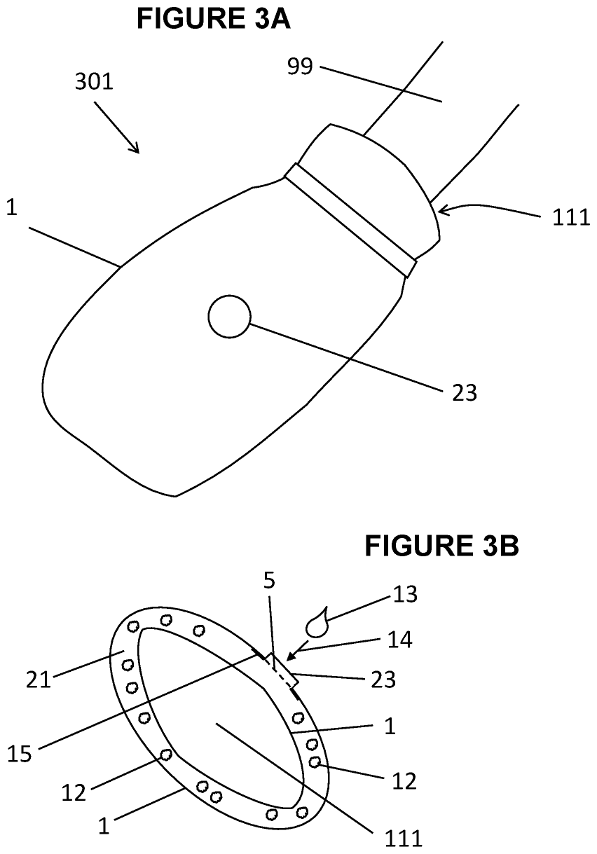

FIG. 3A shows a bag type appendage wearable thermal device 301. An appendage for which the bag type appendage wearable thermal device may be deployed include: hand; hand and arm 99; foot; foot and leg 9; or head. Said bag type appendage wearable thermal device can be similar to a tube with one end closed. The open end of closed tube or bag is appendage insertion opening 111 where an appendage is inserted. A mitten or bag would best describe said bag type appendage wearable thermal device 301 to cover hand. Another type of appendage wearable thermal device may be mitten or bag that includes a section or sleeve going up a selected length of arm 99. Likewise a sock may best describe said bag type appendage wearable thermal device to cover foot. Sock includes a section or sleeve going up and covering a selected length of leg 9. Port assembly 23 is attached to containment pack 1. Port assembly 23 allows for the introduction, storing, and/or sealing of thermally treatable material to be placed into said containment pack 1. Said thermally treatable material may include, but not limited to: water; gel; water and additives such as alcohol, ethanol and others, salt water; rice; oxygen, air, and/or other gases; and/or a thermally reactive material. Fastening device 66 may be hook and loop, and/or a cord and lock mechanism.

FIG. 3B shows a cross section through bag type appendage wearable thermal device 301 of FIG. 3A. Containment pack 1 has appendage insertion opening 111 to receive appendage. Within said containment pack 1 is a selected amount of preloaded component 12. Preloaded component 12 is at least one chemical material needed to initiate or activate a chemical thermal reaction. A thermally treatable material which is a second and needed chemical to initiate or activate said chemical thermal reaction is called introduced component 13. A selected volume of introduced component 13 may be poured through port assembly 23 and/or injected into said containment pack 1 at a selected or desired time. Arrow 14 shows direction of introduced component 13 into containment pack 1. Said preloaded component 12 and introduced component 13 when combined together produce a selected amount of chemical thermal reaction. Said selected chemical thermal reaction may be an endothermic or an exothermic reaction for the purpose of providing: immediate thermal therapy; and/or a measured amount of thermal therapy such as cooling therapy or heating therapy, respectively. An example of an endothermic reaction is: a selected amount of ammonia nitrate is preplaced in containment pack 1 and is called preloaded component 12; then at a selected time, a selected amount of water called introduced component 13 is poured or injected through port assembly 23 into containment pack 1; and when preloaded component 12 and introduced component 13 mix create a selected amount of thermal energy or cooling effect for therapy. Screen 5 may be used, in part, to keep preloaded component 12 from falling out of containment pack 1. An example of an exothermic reaction is iron powder as preloaded component 12 and introduced component 13 may be oxygen or air.

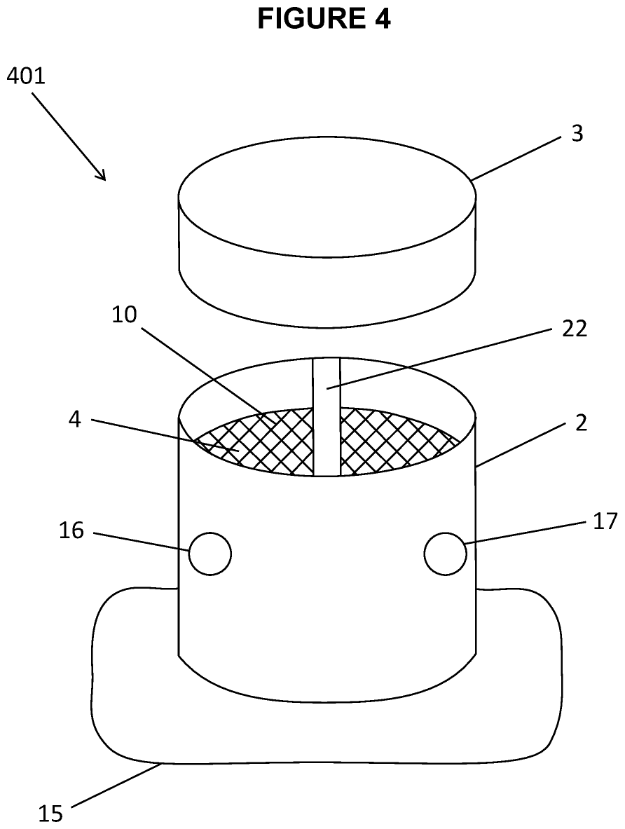

FIG. 4 shows an example of a multiuse port assembly 401. Port body 2 has multiple functions that include, but not limited to: thermally treatable material may be poured into containment pack 1 through port body 2 and port opening 4; and/or thermally treatable material may be introduced into containment pack 1 and port opening 4 through at least one valve 16, 17 where said thermally treatable material is under pressure. Flange 15 is used to join port 2 to containment pack 1 (also see FIG. 3B). Flange 15 may be separately sealed to containment pack with a fitting that would allow port body 2 to be placed. Port divider or port partition 22 divides port body 2 and port opening 4. Both sides of port body 2 and port opening 4 open into volume 21 and at different ends of containment pack 1. Valves 16, 17 are shown to partially protrude into port opening 4 in order to: best describe; and/or provide stronger port structure. Said valves 16, 17 may not protrude into port opening 4, in order to provide larger or a greater port opening 4. Valves 16, 17 may be, but not limited to: drip-less values; leak-less values; pneumatic valves; compression coupling; and/or twist to connect values. A selected version is where port body 2 is permanently sealed, or cap 3 is not necessary as it becomes part of port body 2 with access only through at least one valve. Upper screen 10 may be used to protect valves

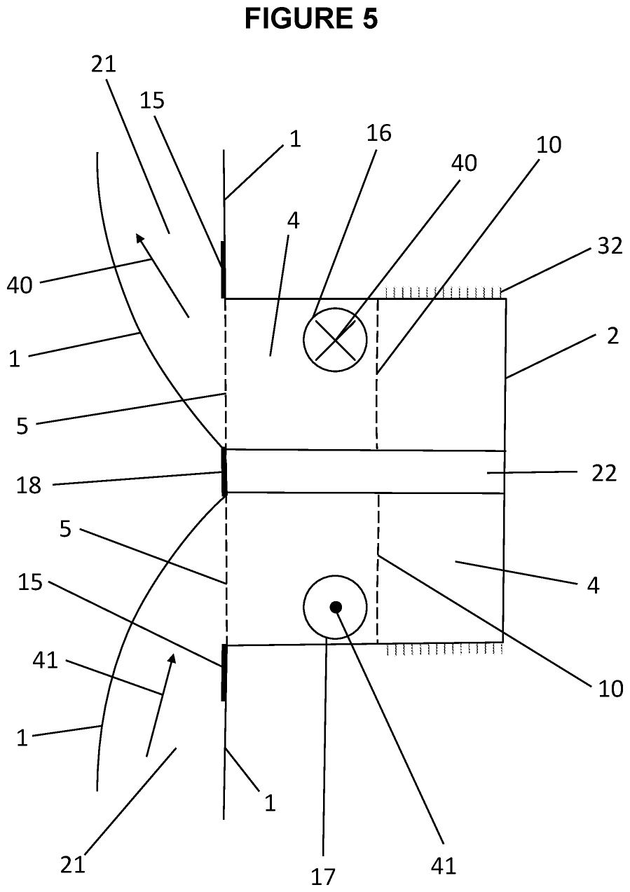

FIG. 5 shows cross sectional view of port body 2 used in multiuse port assembly 401 (see FIG. 4). Port body 2 is sealed or attached to containment pack 1 by: flange 15; and port partition seam 18. Port divider or port partition 22 is sealed to containment pack 1 along port partition seam 18. Valves 16, 17 function to introduce and/or removal of contents, into and out of containment pack 1. Port divider or port partition 22 separates flows going in different or opposite directions; and generally located at each end of containment pack 1 (see FIG. 8, 9). Screen 5 and upper screen 10: protect valves from blockage; prevent debris from entering containment pack 1; and may also be used to project contents of containment pack in a controlled and/or distributed manner. Projection of contents can be achieved by simply squeezing containment pack 1 or wearable thermal device resulting in a spray of mixed and thermally spent preloaded component 12 and introduced component 13 (see FIG. 3B). For example containment pack 1 may contain a mixture of ammonia nitrate and water which is a fertilizer. Novel use of said screen 5 and/or upper screen 10 is to control projection or spray out onto a patch of land much like a shower head for the intent to grow food, thus providing a second use for wearable thermal device. Intake valve 16 may provide for the pressured introduction of: external fluids or gases; introduced component 13 into containment pack 1 to activate desired thermal reactions; and/or an inlet for circulating externally thermally treated material. Valve 17 is use as out take of material in containment pack 1 and in combination with valve 16 to function together to extend thermal treatments by circulating externally thermally treated fluid and/or gas from an external source through containment pack 1 (see Welkins, U.S. Pat. No. 6,551,347). For example, flow into containment pack 1 and/or volume 21 through intake valve 16 is intake valve flow direction 40. Flow out of containment pack 1 and/or volume 21 through outtake valve 17 is outtake valve flow direction 41. Material flow can be controlled by: intake valve 16 is larger than out take valve 17; external thermal machine; and/or flow restrictions using volume deflectors 81 close to out take valve 17. Closing cap 3 goes over port body 2 to close or seal off port opening 4 with a closing mechanism that may be, but not limited to: screw type; quick release coupling; twist and connect coupling; some other sealing mechanism; and/or permanently sealed. Screw type threaded mechanism 32 as in a male screw type is shown and said closing cap having complementary female type treading.

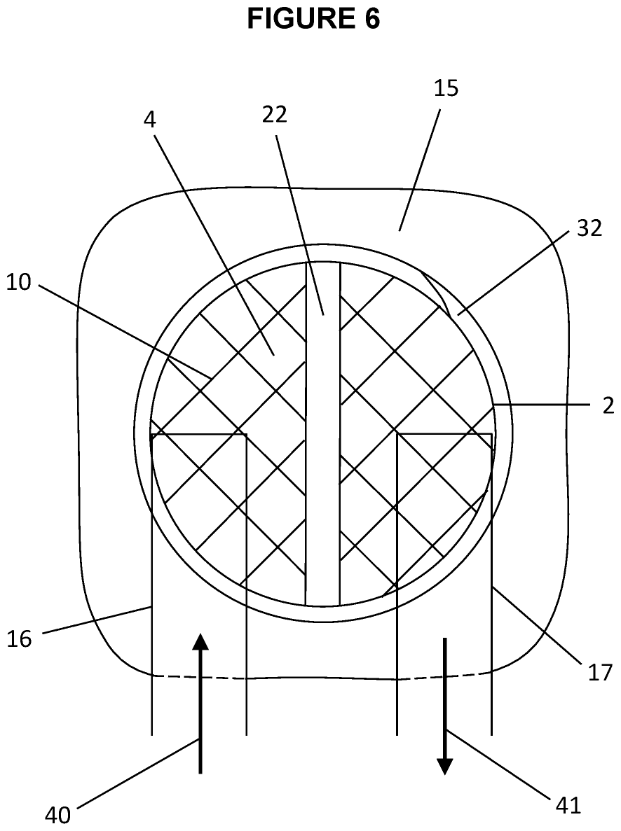

FIG. 6 shows a top view into body of multiuse port body 2 used in multiuse port system 401 (see FIG. 4). Flange 15 is used to seal port body 2 to containment pack 1 and/or wearable thermal device. Closing mechanism or male screw threaded mechanism 32 is used in conjunction with a female screw type closing cap and seals port opening 4. Port divider or partition 22 functions to separate: different ends of containment pack 1; different ends of volume 21; valves 16, 17; and different flow directions 40, 41. Valves 16, 17 may be connected, by hoses, to external source of thermally treated material to extend thermal treatment.

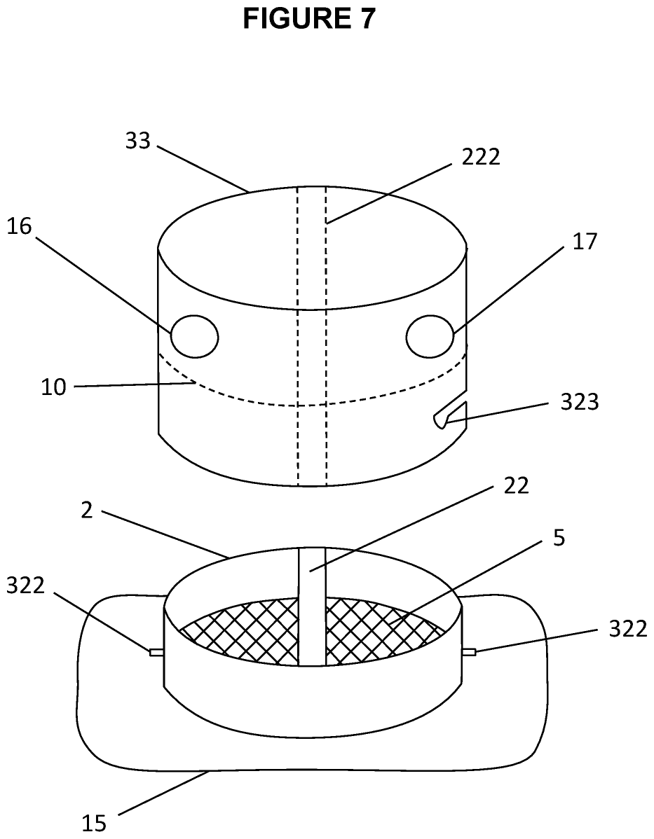

FIG. 7 shows a particular embodiment where some functionality of multiuse port assembly is positioned in port body 2, and others are positioned in closing cap 33. Port body 2 is attached to containment pack 1 with flange 15 and port partition seam 18. Port body 2 may include, but not limited to: port divider or port partition 22; screen 5; and/or port body closing mechanism 322. This configuration of port body 2 allows for the easy and accessible pouring of thermally treatable material into containment pack 1 and volume 21. Valve closing cap 33 consists of at least one valve and contains other functionality of multiuse port. Valve closing cap 33 may include, but limited to: port cap divider or partition 222; upper screen 10; intake valve 16; out take valve 17; and/or cap closing mechanism 323. Port cap divider or partition 222 and upper screen 10 are located within the valve closing cap 33 and because they are hidden from this view they are represented by dashed lines. Port cap divider or partition 222 is intended to: align with port divider or port partition 22 of port body 2; seal both ends of containment pack 1 when attached to port body; and/or separate different flow directions into and out of containment pack 1. At least one cap closing mechanisms 323 may be a press and twist to connect type connector which is intended to mate with at least one port body closing mechanism 322. Normally two closing cap closing mechanism 323 and two port body closing mechanism 322, but there may be more such as three to better secure valve closing cap 33 to port body 2. Upper screen 10 is inside valve closing cap 33 and is therefore represented by dashed lines. Upper screen 10 protects valves from debris associated with objects not passed through screen 5 and remaining in port body 2. Multiuse port functions to allow for the introduction of a selected amount of thermally treatable material and/or thermally treated material through at least one valve. Valves 16, 17 are integrated into valve closing cap 33 assembly and act in the same manner as described above with different flow directions 40, 41

FIG. 8 shows multiuse port in combination with a wrapping type foot wearable thermal device (see FIG. 2) which is intended to wrap around foot and selected portion of leg 9. Multiuse port body 2 is shown with intake valve 16, out take valve 17 and port divider or port partition 22. Intake valve 16 provides intake valve flow direction 40. Out take valve 17 provides for out take flow direction 41. Port partition seam 18 and volume deflectors 81 further assists in separating each end of containment pack 1. Volume deflectors 81 may consist of: sealed lines within outline or selected shape edges of said containment pack 1; selectively sealed lines of limited permeability within outline or selected shape edges of said containment pack 1; and/or separate barriers selectively placed to separate compartments and/or flow channels. Volume deflectors 81: control flow and flow direction 42 between valves 16, 17, where flow direction 42 is indicated by arrows; create thermal compartments, and/or flow channels within containment pack 1; may provide for selected distribution of preloaded component 12 within containment pack 1 (see FIG. 3B); provide control of selected amount of volume 21; and/or control amount of introduced component 13. A particular embodiment of this invention features initial temperature controls and/or thermal mass control. Initial temperature controls and/or thermal mass transfer capability control can be realized through varying the amount of said preloaded component 12 and introduced component 13. Selective volume 21 and a selective temperature of material in said selective volume 21 determines a selected amount of thermal energy or thermal mass. Target temperatures needed or desired for thermal therapeutic application of an appendage or body part is used to determine the amount of thermal mass to be created by designing and using a) selected volume 21 and b) selected temperatures. Temperatures are controlled by specifying the: amount of thermal reaction; amount of preloaded component 12; and/or amount of introduced component 13. Each fastening flap 6 contains a latching material to attach to fastening counterpart 7. Fastening flap 6 and fastening counterpart 7 allow wrapping type foot wearable thermal device to stay wrapped around a foot and/or leg.

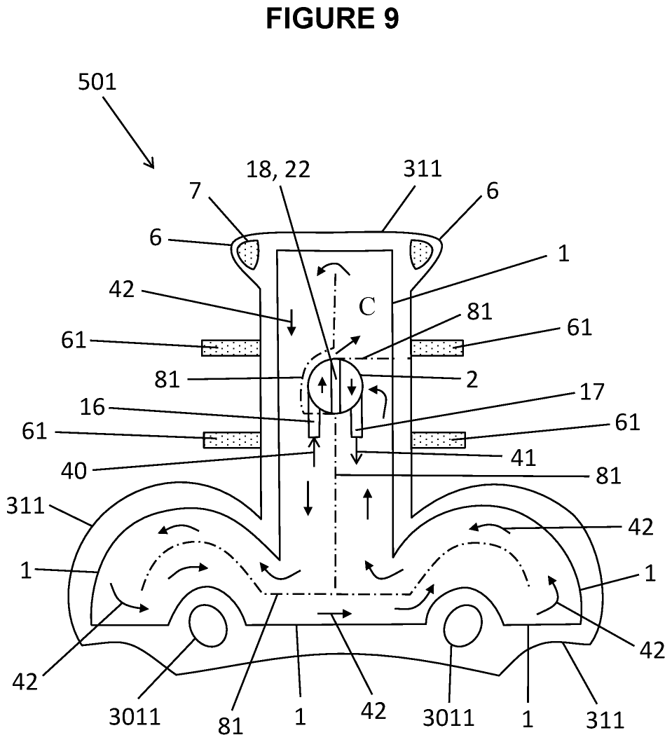

FIG. 9 shows a wrapping, bag type appendage wearable thermal device 501 with one appendage insertion opening that is intended to wrap and close around the head. This particular embodiment comprises a containment pack 1 within wearable thermal device 301. Appendage wearable thermal device 501 has a selected wearable thermal device outline 311 and contains containment pack 1. Containment pack 1 may be inserted into and/or attached to appendage wearable thermal device 501 and may have layers. Layers are functional and include, but not limited to: thermal insulation; radiant barrier; sensors; and/or skin protectant. Said appendage wearable thermal device uses fastening mechanism strap 61 and fastening counterpart 7, which may be hook and loop, or other fastening system to close appendage wearable thermal device 501 around a head. FIG. 9 intended view is from the inside of wearable thermal device hence fastening counterpart 7 associated with fastening mechanism strap 61 are not shown. Port body 2 of multiuse port is shown with intake valve 16, out take valve 17, and port divider or port partition 22. Port divider or port partition 22 is located in the same position as port partition seam 18, in this view. Valve 16, 17 provide access to both ends of containment pack 1 and are separated by: port partition seam 18; port divider or port partition 22; and volume deflectors 81. Intake valve 16 provides intake valve flow direction 40. Intake valve 16 can be used to: fill containment pack 1 with thermally treatable material; fill containment pack 1 with introduced material 13; and/or add external thermally treated fluid and/or gas into containment pack 1 or volume 21. Intake valve 16 may be used to transport introduced component 13 into containment pack 1 or volume 21 to mix with preloaded component 12 (see FIG. 3B) to activate a thermal reaction. Flow direction 42 within containment pack 1 or volume 21 is shown as arrows. Volume deflectors 81, shown as dashed lines, control flow direction 42 to ensure even distribution and coverage of thermal therapeutic treatment. Volume deflectors 81 may also function to control volume 21, create thermal compartments C, and/or shape of containment pack 1. An example of a thermal compartment C within volume 21 is a larger pool of thermal material in the left frontal brain lobe of the wrapping, bag type appendage wearable thermal device 501. Out-take valve 17 may provide out take flow 41 out of said containment pack 1 and/or volume 21. A particular embodiment of current invention is the ability to extend emergency treatment in a medical event through the use of multiuse port and external thermal treating machine. Extended thermal treatments may last for extended periods of time, perhaps for days, weeks or months. Ear cutouts are provided for sound transmission holes 3011.

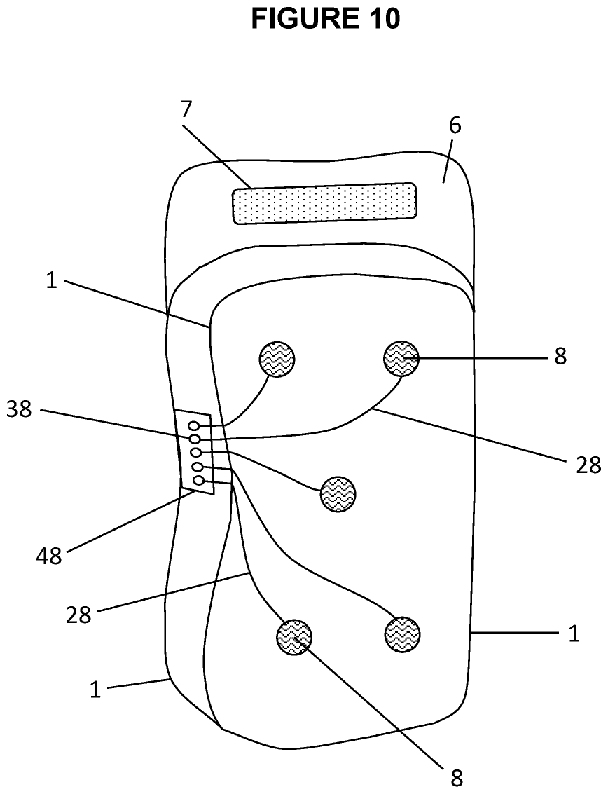

FIG. 10 shows wearable thermal device with biosensors 8. Biosensors 8 that may be used in and/or on wearable thermal device may include: but not limited to: temperature sensors; sensors for body part; electrical sensors as in a EEG, electroencephalogram; and/or, optical sensors as in PPG, photoplethysmography. It is the intent of the current invention to provide a multitude of sensors in a layer to measure appendage or body part physical and biological characteristics. Temperature sensors may be simple temperature strips that display color indications of temperature of device, or connected to a display unit for more accurate reading. Other temperature sensors may monitor body part temperature. Biosensor wires 28 connect biosensors 8 to biosensor port 48 where each sensor has its own connection or channel 38. Note that in FIG. 10, this particular configuration, said port body 2 is on opposite side of containment pack 1 and out of view. Multiuse port may be integrated with biosensor port 48 to provide additional capabilities into multiuse port. An advantage is a multiuse port can now provide patient and device monitoring capability as well as thermal treatments. Biosensor port 48 may include a multiplexer. Said multiplexer may reduce: cost; minimize wire between device and external signal collection unit; and/or reduce weight of device on patient. Fastening flap 6 and fastening counterpart 7 may be a hook or loop material. An example is a wearable device containing multiple PPG sensors with the intent to measure and display a 3 dimensional analysis of; blood flow, blood pressure, cellular pressure, and/or oxygen levels. Biosensors 8 may be gridded in a selected shape, configuration, density and/or pattern to analysis biological characteristics.

1. A wearable thermal device comprising:
a containment pack of selected shape;
said containment pack contains a selected amount of first material;
at least one port is attached to said containment pack configured to accept a second material; and
said second material is configured to be added through said at least one port to create a chemical reaction when combined with said selected amount of first material.

2. The wearable thermal device of claim 1 wherein said at least one port has at least one screen.

3. (canceled)

4. The wearable thermal device of claim 1 wherein said at least one port has at least one valve.

5. The wearable thermal device of claim 1 has at least one sensor.

6. The wearable thermal device of claim 1 has at least one insulation layer.

7. A wearable thermal device of claim 1 wherein a layer of sensors around appendage or body part provides three dimensional imaging.

8. A target temperature wearable device comprising:
a containment pack of selected shape and selected volume;
said containment pack contains a selected amount of first material;
at least one port is attached to said containment pack configured to accept a selected amount of second material;
said second material is configured to be added through said at least one port to create a selected amount of thermal mass when combined with said selected amount of first material; and
said at least one port has a closing cap.

9. The target temperature wearable device of claim 8 where said at least one port consists of at least one screen.

10. The wearable thermal device of claim 1 where said at least one containment pack has a selected volume configured to accept a selected amount of said second material when combined with said selected amount of first material create a selected amount of thermal mass.

11. An extended wearable device of claim 8 where at least one port consists of at least one valve.

12. An extended wearable device of claim 8 where at least one port consists of at least one intake valve and at least one outtake valve.

13. (canceled)

14. The target temperature wearable device of claim 8 has at least one temperature sensor.

15. A multiuse port assembly comprising:
at least one screen;
at least one valve;
at least one partition; and
a flange to connect to a containment pack.

16. A multiuse port as in claim 15 comprises sensor junction, said sensor junction houses connections from onboard sensors.

17. (canceled)

18. A multiuse port assembly as in claim 15 further consisting of a multiplexer.

19. An extended targeted temperature wearable device comprising:
a containment pack of selected shape;
said containment pack contains a selected amount of first material;
at least one port has at least one valve attached to said containment pack configured to accept a second material; and
said second material is configured to be added through said at least one valve to create a selected amount of thermal mass when combined with said selected amount of first material.

20. The extended target temperature wearable device of claim 19 has at least one biosensor.

21. The extended target temperature wearable device of claim 19 where said at least one port further includes at least one outlet valve.

22. The extended target temperature wearable device of claim 19 is connected to an external thermal conditioning machine.

23. The extended target temperature wearable thermal device of claim 19 has at least one layer.

24. The wearable thermal device of claim 1 has at least one radiant barrier layer.

25. The wearable thermal device of claim 1 has at least one skin protectant layer.

26. The wearable thermal device of claim 1 has at least one fastener.

27. The wearable device of claim 1 where said at least one port has a closing cap.

28. The target temperature wearable device of claim 8 has at least one layer.

29. The wearable thermal device of claim 1 further consists of more than one containment pack.
